(19) United States
(12) Patent Application Publication
(10) Pub. No.: US 2025/0380996 A1
(43) Pub. Date: Dec. 18, 2025

(54) FORCE TRANSMISSION SYSTEMS FOR INSTRUMENTS, AND RELATED DEVICES

(71) Applicant: INTUITIVE SURGICAL OPERATIONS, INC., Sunnyvale, CA (US)

(72) Inventors: Iman Khalaji, Santa Clara, CA (US); Scott E. Manzo, Shelton, CT (US)

(73) Assignee: INTUITIVE SURGICAL OPERATIONS, INC., Sunnyvale, CA (US)

(21) Appl. No.: 18/877,558

(22) PCT Filed: Jun. 20, 2023
(86) PCT No.: PCT/US2023/068698

§ 371 Date: Dec. 20, 2024

Related U.S. Application Data
(60) Provisional application No. 63/353,978, filed on Jun. 21, 2022.

(51) Int. Cl. A61B 34/30; A61B 34/00

(52) U.S. Cl. CPC A61B 34/30; A61B 34/71; A61B 2034/306

(57) ABSTRACT

An instrument includes a shaft comprising a distal end portion, a proximal end portion, and a longitudinal axis extending between the distal end portion and the proximal end portion, and a moveable component coupled to the distal end portion of the shaft. A drive assembly is coupled to the proximal end portion of the shaft, the drive assembly comprising a gimbal assembly rotatable about a first axis and a second axis. A first actuation member is coupled to the gimbal assembly and extends from the drive assembly along the shaft and coupled to the moveable component, and a second actuation member is coupled to the gimbal assembly and extends along the drive assembly along the shaft and coupled to the moveable component. Devices, systems, and methods relate to gimbal assemblies and related drive assemblies.

### CROSS-REFERENCE TO RELATED APPLICATION

This application claims priority to U.S. Provisional Application No. 63/353,978 (filed Jun. 21, 2022), titled “FORCE TRANSMISSION SYSTEMS FOR INSTRUMENTS, AND RELATED DEVICES AND METHODS” the entire contents of which are incorporated by reference herein.

### TECHNICAL FIELD

Aspects of the present disclosure relate to force transmission systems and related devices and methods. For example, aspects of the present disclosure relate to force transmission systems that convert rotational input forces to translational forces that can be transmitted along an instrument to actuate components of the instrument.

### INTRODUCTION

Various medical (including surgical) and industrial instruments include shafts and have one or more components that impart one or more degrees of freedom of movement to such instruments. Such components can be in the form of end effectors that move in one or more degrees of freedom, such as for example, translating mechanisms, jaws that open and close, etc. Other such components may include articulable structures, such as joint mechanisms along the shaft that are pivotable (e.g., in pitch and/or yaw) relative to the shaft. These articulatable structures can couple an end effector to the shaft and/or couple a relatively proximal portion of the shaft to a relatively distal portion of the shaft. These components that impart one or more degrees of freedom to the instrument can be actuated and controlled via translating actuation members extending along a length of the shaft. Such actuation members may be in the form of pullable (tension) members such as cables, wires, filaments or the like that are flexible in all directions and generally transmit stronger force by pulling on the actuation members to place it in tension (sometimes referred to as pull-pull actuation members); more rigid members such as tubes, rods, sheet metal strips, or the like that can transmit force by pushing or by pulling on the actuation members (sometimes referred to as push-pull actuation members); semi-flexible pushing members such as push coils that can transmit force by pushing while providing lateral flexibility; rotatable members such as leadscrews that can transmit rotational force; and a variety of other forms of actuation members. The actuation members extend through the instrument shaft to couple to an actuatable component (e.g., articulatable structure and/or a moveable end effector component) at a relatively distal portion of the shaft and to a drive member at a force transmission system at a relatively proximal portion of the instrument shaft. In this way, the actuation members transmit forces from the force transmission system, which can remain at a remote location from the work site (e.g., outside a patient's body in the case of a medical instrument performing a medical procedure) to the actuatable component, which is proximate a worksite (e.g., inside a patient's body in the case of a medical instrument performing a medical procedure). Force transmission systems can have manually-operated inputs for instruments that are manually operated or can include input interfaces that are configured to engage with a manipulator system of a teleoperated, computer-assisted system, which manipulator systems comprise motorized output drives that are under control from remote input mechanisms, as would be familiar to those of ordinary skill in the art.

In some force transmission systems, the drive members to which pull-pull type actuation members are coupled are rotary drive members, such as a rotating drum (e.g., capstan), a rotating shaft, or a pulley. More specifically, the actuation members are coupled to the rotary drive members and rotary motion causes the actuation members to be paid in (partially wound around the rotary drive member) and paid out (partially unwound from the rotary drive member) to transmit force to the actuatable component. Moreover, depending on the arrangement of the force transmission system, the actuation members may be required to follow relatively complex paths to their coupling with the drive member, such as being routed around one or more pulleys or other routing mechanisms to a drive member of the force transmission system. These mechanisms may limit the possible types of actuation members that can be used to the pull-pull type actuation members, such as cables, wires, other filament structures, or the like, so as to provide the flexibility needed to follow more circuitous paths to the ultimate rotary drive member.

In addition, to reduce backlash and facilitate accurate movement and control of the actuatable components, particularly articulable structures for example, it is sometimes desired to pre-tension such pull-pull type actuation members during manufacturing of the instrument to remove slack that would otherwise lead to inaccuracies in movement and positioning of the actuatable component. Such pre-tensioning can introduce additional complexity to the manufacturing process, particularly with instruments that include directional changes of the actuation members, e.g., around pulleys, capstans, or other routing mechanisms within the force transmission system, as discussed above.

Further, the use of multiple actuation members to control multiple degrees of freedom of one or more actuatable components can further complicate manufacture of the instruments. In particular, the routing and operable coupling of multiple actuation members poses challenges in attempting to automate manufacturing of the instruments due to the many routing paths and connections that may be needed.

There exists a need for force transmission systems that simplify and facilitate manufacturing, reduce overall part count, and that provide robust and reliable force transmission for the actuation of actuatable components of instruments. In particular, there exists a need to provide force transmission systems and their corresponding actuation members that may enable more automated manufacturing of instruments, while providing the durability and force transmission properties that allow for input torque to be received by the drive members at the force transmission systems and converted to linear actuation forces of actuation members that can be used to impart sufficiently large force to actuatable components, such as those at the end effector and/or articulable structures.

### SUMMARY

Embodiments of the present disclosure may solve one or more of the above-mentioned problems and/or may demonstrate one or more of the above-mentioned desirable features. Other features and/or advantages may become apparent from the description that follows.

In accordance with at least one aspect of the present disclosure, an instrument comprises a shaft comprising a distal end portion, a proximal end portion, and a longitudinal axis extending between the distal end portion and the proximal end portion. A moveable component is coupled to the distal end portion of the shaft and a drive assembly coupled to the proximal end portion of the shaft. A first actuation member extends from the drive assembly along the shaft and is coupled to the moveable component. A second actuation member extends from the drive assembly along the shaft and is coupled to the moveable component. The drive assembly comprises a gimbal assembly rotatable about a first axis and a second axis, the gimbal assembly comprising a first aperture and a second aperture. The first actuation member is routed through the first aperture and coupled to the gimbal assembly, the first actuation member being longitudinally translatable relative to the shaft in response to rotation of the gimbal assembly about the first axis. The second actuation member is routed through the second aperture and coupled to the gimbal assembly, the second actuation member being longitudinally translatable relative to the shaft in response to rotation of the gimbal assembly about the second axis. The first aperture defines an elongated opening in a direction perpendicular to the second axis, and the second aperture defines an elongated opening in a direction perpendicular to the first axis.

In another aspect of the present disclosure, an instrument comprises a shaft comprising a distal end portion, a proximal end portion, and a longitudinal axis extending between the distal end portion and the proximal end portion. A moveable component is coupled to the distal end portion of the shaft. A drive assembly is coupled to the proximal end portion of the shaft, and the drive assembly comprises a gimbal assembly rotatable about a first axis and a second axis. A first actuation member is coupled to the gimbal assembly and extends from the drive assembly along the shaft and is coupled to the moveable component. A second actuation member is coupled to the gimbal assembly and extends along the drive assembly along the shaft and is coupled to the moveable component. In response to rotation of the gimbal assembly about the first axis, the first actuation member is linearly translatable relative to shaft and the second actuation member remains substantially stationary, and in response to rotation of the gimbal assembly about the second axis, the second actuation member is linearly translatable relative to shaft and the first actuation member remains substantially stationary.

In yet another aspect of the present disclosure, an instrument comprises a shaft comprising a proximal end portion and a distal end portion. A moveable component is coupled to the distal end portion of the shaft, and a force transmission system coupled to the proximal end portion of the shaft. A first actuation member extends from the force transmission system along the shaft and is coupled to the moveable component, the first actuation member configured to transmit force from the force transmission system to the moveable component. A second actuation member extends from the force transmission system along the shaft and is coupled to the moveable component, the second actuation member configured to transmit force from the force transmission system to the moveable component. The force transmission system comprises a gimbal rotatable about a first gimbal axis and a second gimbal axis, a first lever arm comprising a first end portion and a second end portion operably coupled to the gimbal, the first lever arm being configured to rotate about a first pivot axis between the first end portion and the second end portion of the first lever arm, and a second lever arm comprising a first end portion a second end portion operably coupled to the gimbal, the second lever arm being configured to rotate about a second pivot axis between the first end portion and the second end portion of the second lever arm. In response to pivoting of the first lever arm about the first pivot axis, the gimbal rotates about the first gimbal axis. In response to pivoting of the second lever arm about the second pivot axis, the gimbal rotates about the second gimbal axis. Rotation of the gimbal about the first gimbal axis results in linear translation of the first actuation member along the shaft, and rotation of the gimbal about the second gimbal axis results in linear translation of the second actuation member along the shaft.

In yet another aspect of the present disclosure, an instrument comprises a shaft comprising a distal end portion and a proximal end portion, an end effector coupled to the distal end portion of the shaft, a drive assembly at the proximal end portion of the shaft, and an actuation member extending through the shaft and coupled to the drive assembly and the end effector, the actuation member configured to transmit force from the drive assembly to the end effector. The drive assembly comprises a rotatable drive shaft operably coupled to an input drive member, a pinion gear coupled to the rotatable drive shaft, and a driven gear assembly comprising a driven gear and a retention feature configured to be removably engageable with the pinion gear. In a state of engagement of the pinion gear and the retention feature, the driven gear is operably coupled to the pinion gear and the actuation member such that rotation of the driven gear via the pinon gear results in linear translation of the actuation member.

In yet another aspect of the present disclosure, an instrument comprises a shaft comprising a distal end portion, a proximal end portion, and a longitudinal axis extending between the distal end portion and the proximal end portion, a moveable component coupled to the distal end portion of the shaft, an end effector coupled to the movable component, the end effector comprising one or more movable jaws and a translatable cutting element, a drive assembly coupled to the proximal end portion of the shaft, a first actuation member extending through the shaft and coupled to the drive assembly and the end effector, the first actuation member configured to transmit force from the drive assembly to operate the jaw of the end effector, and a second actuation member extending through the shaft and coupled to the drive assembly and the end effector, the second actuation member configured to transmit force from the drive assembly to operate the translatable cutting element of the end effector. The drive assembly comprises a first rotatable drive shaft operably coupled to a first input drive member, a first pinion gear coupled to the first rotatable drive shaft, and a first driven gear assembly comprising a first driven gear and a retention feature configured to be removably engageable with the first pinion gear. In a state of engagement of the first retention feature and the first pinion gear, the first driven gear is operably coupled to the first pinion gear and the first actuation member such that rotation of the first driven gear via the first pinon gear results in linear translation of the first actuation member and actuation of the one or more movable jaws. The drive assembly further comprises a second rotatable drive shaft operably coupled to a second input drive member, a second pinion gear coupled to the second rotatable drive shaft, and a second driven gear assembly comprising a second driven gear and a second retention feature configured to be removably engageable with the second pinion gear. In a state of engagement of the second retention feature and the second pinion gear, the second driven gear is operably coupled to the second pinion gear and the second actuation member such that rotation of the second driven gear via the second pinon gear results in linear translation of the second actuation member and actuation of the translatable cutting element. The drive assembly further comprises a gimbal assembly rotatable about a first axis and a second axis, a third actuation member coupled to the gimbal assembly and extending from the drive assembly along the shaft and coupled to the moveable component, and a fourth actuation member coupled to the gimbal assembly and extending along the drive assembly along the shaft and coupled to the moveable component. In response to rotation of the gimbal assembly about the first axis, the third actuation member is linearly translatable relative to shaft and the fourth actuation member remains substantially stationary, and in response to rotation of the gimbal assembly about the second axis, the fourth actuation member is linearly translatable relative to shaft and the third actuation member remains substantially stationary.

Additional objects, features, and/or advantages will be set forth in part in the description which follows, and in part will be obvious from the description, or may be learned by practice of the present disclosure and/or claims. At least some of these objects and advantages may be realized and attained by the elements and combinations particularly pointed out in the appended claims.

It is to be understood that both the foregoing general description and the following detailed description are for example and explanatory only and are not restrictive of the claims; rather the claims should be entitled to their full breadth of scope, including equivalents.

### BRIEF DESCRIPTION OF THE DRAWINGS

The present disclosure can be understood from the following detailed description, either alone or together with the accompanying drawings. The drawings are included to provide a further understanding of the present disclosure and are incorporated in and constitute a part of this specification. The drawings illustrate one or more embodiments of the present teachings and together with the description explain certain principles and operation. In the drawings,

FIG. 1 is a schematic, side view of an embodiment of an instrument comprising a force transmission system according to various embodiments of the present disclosure.

FIG. 2 is a perspective view showing various internal components of a force transmission system according to an embodiment of the present disclosure.

FIG. 3 is a perspective view of an embodiment of a gimbal drive assembly according to an embodiment of the present disclosure.

FIG. 4 is a top view of the gimbal drive assembly of FIG. 3.

FIGS. 5A, 5B, and 5C are perspective views of drive components of the force transmission system of FIG. 2.

FIG. 6 is a partial perspective view of a gimbal drive assembly according to the present disclosure.

FIGS. 7A through 7E are schematic side and perspective views of actuation members according to the present disclosure and shown in various configurations.

FIG. 8 is a perspective view of a gimbal plate according to an embodiment of the present disclosure.

FIG. 9 is a cross-sectional view of the gimbal plate of FIG. 8 taken through section 9-9.

FIGS. 10A and 10B are perspective views of various components of the end effector drive system of claim 2 shown in isolation.

FIG. 11 is a perspective view of other components of the end effector drive system of claim 2 shown in isolation.

FIG. 12 is a perspective, partial view of an end effector assembly according to an embodiment of the present disclosure.

FIG. 13 is a perspective schematic view of a manipulator system according to some embodiments of the present disclosure.

FIG. 14 is a partial schematic view of another embodiment of a manipulator system according to some embodiments of the present disclosure.

### DETAILED DESCRIPTION

Embodiments of the present disclosure relate to instruments and associated force transmission systems that are configured to drive actuation of actuation members that transmit force to actuate actuatable components, such as for example articulable structures and/or end effectors, coupled at relatively distal portions of the shafts of the instruments. In various embodiments, the force transmission systems are configured to further be operably coupled with drive interfaces of manipulators, such as computer-controlled (e.g., teleoperated) or manual (e.g., laparoscopic) manipulators. Force transmission systems and actuation member drive assemblies of such force transmission systems can, according to various embodiments contribute to ease of manufacturing, e.g., by facilitating use of automated manufacturing processes. Moreover, they can provide for less complexity in operably coupling actuation members to drive assemblies, such as for example, by reducing or eliminating the use of pulleys, capstans, and other drive and routing mechanisms found in drive assemblies that are configured for use with pullable type actuation members and that may require wrapping and relatively complex routing of the actuation members. Further, in some embodiments, actuation members capable of transmitting both tensile and compressive forces (e.g., “push-pull” type actuation members) can be used and can reduce the total number of actuation members needed to articulate a given number of degrees of freedom.

Various embodiments of the disclosure include an actuation member drive assembly including a gimbal (such as a gimbal assembly including a gimbal plate and a gimbal frame) configured to pivot about one or more non-parallel axes, for example, independently and in combination, such as independent axes. This motion of the gimbal enables actuation members operably coupled to the gimbal assembly to be translated linearly, which in turn can be operably coupled to actuate an articulatable structure such as a wrist mechanism in separate degrees of freedom, such as pitch and yaw. In other embodiments, the gimbal can be configured to actuate one or more components of an end effector, other articulatable structures, and other devices without limitation. In an embodiment of the disclosure in which the gimbal is configured to actuate pitch and yaw of a wrist mechanism, the gimbal can be configured to operate at least two pairs of actuation members, one pair of which are operably coupled to the gimbal to actuate the wrist mechanism in one of the two degrees of freedom (e.g. pitch or yaw), and the other pair of which are operably coupled to the gimbal to actuate the wrist mechanism in the other of the two degrees of freedom (e.g., the other of the pitch or yaw). In some embodiments, the pairs of actuation members are pullable actuation members.

In some instruments, articulable structures, such as wrist mechanisms that couple an end effector to an instrument shaft, for example, are configured to provide length conservation of the actuation members associated with each degree of freedom. Stated another way, in a wrist mechanism in which a pair of pull-pull actuation members are associated with a first degree of freedom, e.g., yaw, the wrist mechanism is configured such that for a given articulation in yaw, one actuation member pays in a certain distance and the other actuation member pays out an equal amount. However, due to the kinematics inherent in a gimbal assembly, it is difficult to achieve full length conservation. Additionally, while some wrist mechanisms can include several individual joints, and thus pairs of actuation members, to achieve a total desired range of articulation motion over the entire wrist mechanism, thereby requiring a smaller overall range of motion of each individual joint and associated actuation members operably coupled to actuation each individual joint, it is generally desirable to provide a required range of motion with fewer individual joints so as to reduce the overall number of components, friction, and space requirements, for instance. Thus, in some instances, a single joint for yaw movement and a single joint for pitch movement may be utilized. Obtaining a total desired overall range of motion in a given degree of freedom (e.g., pitch or yaw) with a single joint can exacerbate length conservation issues associated with using a gimbal assembly as part of the drive assembly. For example, the farther from a neutral position the gimbal assembly pivots, the more non-length conservative the actuation members become due to the inherent kinematics. Because each joint requires a greater motion of the gimbal when fewer joints are used to achieve the same overall range of motion, greater differences in length of the controlling pair of actuation members likewise can occur. Such changes in relative length can result in excess slack in one of the actuation members of a pair. In some cases, such as when the joint is loaded, the excess slack can result in dislocation of the joint.

Embodiments of the present disclosure include gimbal assemblies configured to achieve a relatively high degree of length conservation over a greater range of motion of the gimbal assembly as compared to prior designs. For example, in some embodiments, a plane of a gimbal plate from which the actuation members exit is offset from pivot axis of the gimbal assembly about which the gimbal plate pivots to actuate the actuation members. The offset modifies the kinematic characteristics of the gimbal assembly and provides improved conservation of length relative to conventional gimbal designs. In some embodiments, the plane from which the actuation members exit is offset from the pivot axis of the gimbal assembly by providing the gimbal plate with a thickness sufficient to offset the plane by a desired amount. In other embodiments, the gimbal plate can optionally be provided with various surface features that modify the geometry of the gimbal plate at the locations the actuation members exit relative to other portions of the gimbal plate.

In some cases, the thickness of the gimbal plate can result in unwanted motion of the actuation members associated with one degree of freedom when the gimbal assembly is actuated in connection with another degree of freedom. The gimbal assembly can further include features configured to prevent movement of the gimbal plate associated with causing actuation members to actuate a component in one degree of freedom when otherwise moving the gimbal plate in a manner associated with causing other actuation members to actuate the component (or another component) in another degree of freedom. For example, the gimbal plate can include features configured such that actuation member exits in the plate are offset from the pivot axes of the gimbal plate by differing amounts for different orientations of the gimbal plate. Such features can include reliefs, slots, and other features having various shapes as will be discussed in further detail herein.

Advantages of actuation member drive assemblies in accordance with various embodiments compared to other arrangements including various capstans, rotatable shafts, pulleys, and/or other routing mechanisms can include, without limitation, a relatively lower part count, improved manufacturability, and increased reliability, e.g., due to the presence of fewer components and fewer associated failure modes.

Additionally, the actuation member drive assemblies according to various embodiments can include other features to improve manufacturability and decrease overall part count. As discussed above, the gimbal assembly can be configured to control actuation of an articulable structure, such as a wrist mechanism. Other instrument degrees of freedom, such as, for example, grip of a gripping instrument, movement of a cutting blade, shaft roll, or other degrees of freedom, can be actuated by other components in the actuation member drive assembly. For example, grip, cut, and other moveable components of an end effector of the instrument can be controlled by one or more leadscrew arrangements, bellcrank arrangements, or other drive components. These components of the actuation member drive assembly can include features similarly configured to reduce part count, facilitate manufacturability, and maintain a robust and durable instrument. Such features can include bearing and/or shaft carriers separate from, but configured to be fixedly attached to, a main chassis portion of the actuation member drive assembly. Other such features can include resilient retaining portions of input drive members such as shafts, the resilient retaining portions being configured to couple the component comprising the resilient retaining portion to a carrier with which the drive member is configured to be coupled. Such arrangements can likewise reduce an overall part count of the instrument, simplify manufacturing, and otherwise contribute to overall cost efficiency of the instrument, while also achieving a durable and robust instrument capable of transmitting force under relatively large loads.

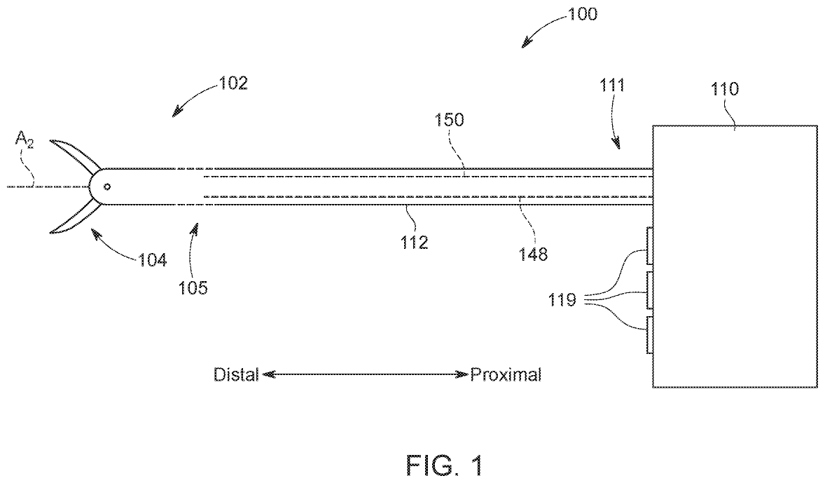

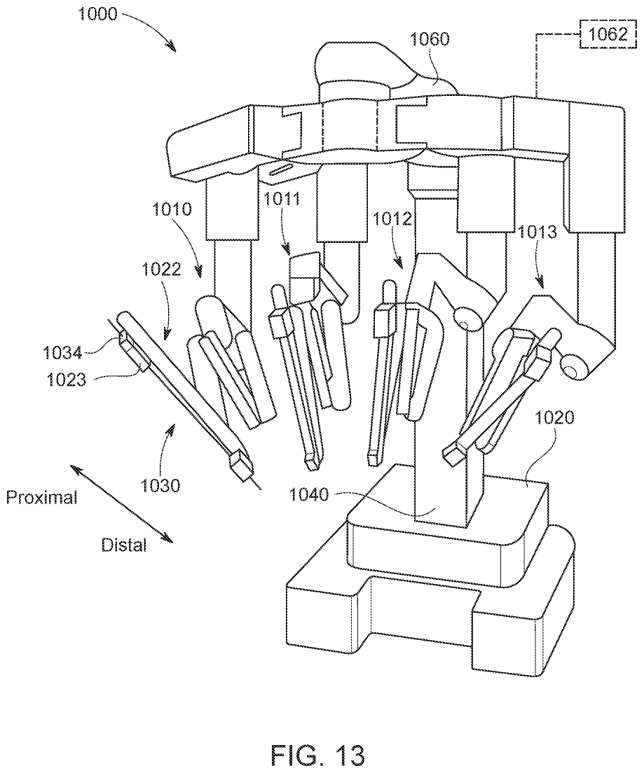

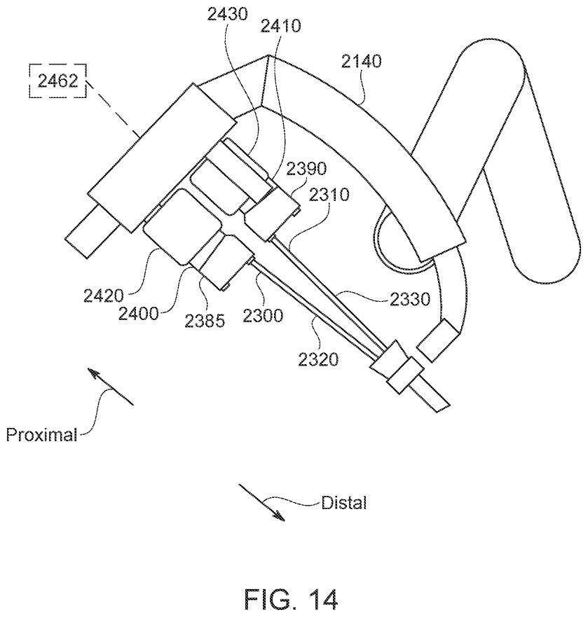

Referring now to FIG. 1, a schematic, side view of an instrument 100 according to some embodiments of the disclosure is shown. Instrument 100 can be or include an instrument used to perform medical (e.g., surgical, diagnostic, and/or therapeutic) or non-medical procedures (e.g., industrial inspection applications). The instrument 100 includes a shaft 112 elongated along a longitudinal axis A_{L}, between proximal end portion 111 and distal end portion 102. The instrument 100 further includes an end effector 104 coupled to the distal end portion 102 and a force transmission system 110 (only the exterior housing portion of which is depicted) coupled to the proximal end portion 111. The end effector 104 is configured to carry out a medical or non-medical (such as industrial) procedure. For example, the end effector 104 can include one or more tools such as gripping tools, staplers, shears, ligation clip appliers, electrosurgical tools, ultrasonic tools, suturing tools, translating sleds, translating cutting tools, or other types of tools. While the illustration of FIG. 1 depicts an end effector 104 having jaw members configured to move toward and away from each other (either by one or both jaw members pivoting about a pivot axis A_{P}), such a configuration is exemplary and non-limiting and those of ordinary skill in the art would appreciate the instrument 100 can have any of a variety of end effectors without departing from the scope of the present disclosure. In the embodiment of FIG. 1, the force transmission system 110 is coupled to the proximal end portion 111 of the shaft 112. In other embodiments, the force transmission system 110 may be coupled at various locations along the shaft 112, and in some cases moveable along the shaft, but generally in a position such that it remains external to a remote site (such as a patient's body) at which the end effector 104 and a distal end portion 102 of the shaft 112 are inserted to perform a procedure, thereby permitting access to manipulate inputs on the force transmission system 110. The force transmission system 110 can be configured to be operably coupled with a computer-controlled (e.g., teleoperated) surgical manipulator system, such as the manipulator systems described in further detail below in connection with FIGS. 13 and 14 or similar manipulator systems with which those having ordinary skill in the art are familiar. For example, the force transmission system 110 can be configured to interface with the drive output assembly 1023 of the manipulator system discussed in connection with FIG. 13, or drive assemblies 2420 and 2430 discussed in connection with FIG. 14, such as via rotary input drive discs 119. In other embodiments, in addition to or in lieu of being configured to interface and be driven by a computer-assisted manipulator system, the force transmission system 110 can be manually controlled with manually-operated (e.g., handheld) manipulators, such as triggers, wheels, buttons, joysticks or the like (not shown).

In the embodiment shown in FIG. 1, the instrument 100 includes an articulable structure 105 coupling the end effector 104 to the shaft 112. As shown in FIG. 1, the articulable structure 105 can be positioned along the distal end portion 102 of the shaft 112. But the disclosure is not so limited and the articulable structure 105 can be positioned at any location along the shaft 112 without limitation. In addition, the instrument 100 can include more than one articulable structure 105, such as two, three, or more articulable structures located at multiple spaced apart locations along the length of the shaft 112. The articulable structure 105 can be controlled and actuated via actuation members, two of which 148 and 150 are illustrated, such as cables, rods, or other structures (shown in dashed lines in FIG. 1; further discussed in connection with the various embodiments disclosed herein) operably coupled to one or more drive components of the force transmission system 110, and thus able to be actuated via a manipulator through the force transmission system. The articulable structure 105 can include one or more joints configured to pivot or flex (e.g., in the case of a continuously flexible joint) relative to the shaft 112. In various embodiments, as those having ordinary skill in the art would be familiar with, an articulable structure can serve as a wrist mechanism supporting and coupling the end effector 104 to the shaft 112 so as to allow orientation of the end effector 104 relative to the shaft in pitch and/or yaw.

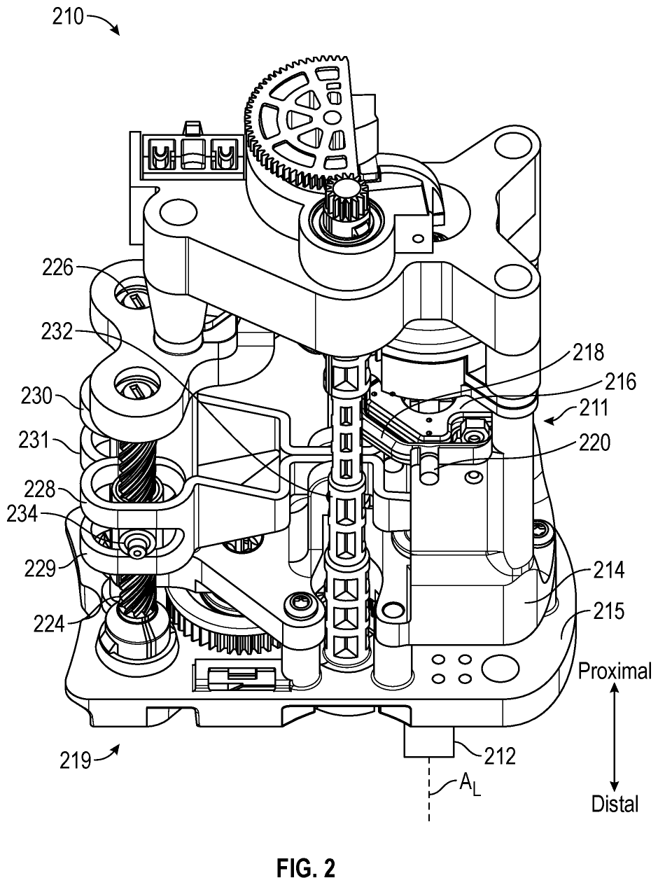

Referring now to FIG. 2, a force transmission system 210 according to various embodiments that can be used as force transmission system 110 is shown. In the embodiment of FIG. 2, the force transmission system 210 is shown with a housing cover of the system removed to better illustrate interior components. As shown in FIG. 2, the force transmission system 210 includes a base 215 and a support chassis 214 to which various components of the force transmission system 210 are coupled. A shaft 212 (e.g., corresponding to shaft 112 in FIG. 1) extends distally from the force transmission system 210 along a longitudinal axis A_{L}.

The force transmission system 210 includes input devices configured to receive input from a manipulator, such as a manipulator that operates with computer assistance (e.g., part of a teleoperated, robotic manipulator system) or a manual manipulator, as noted above. The input devices can be or include, for example, rotary input drive discs 119 (shown in FIG. 1) or other coupling features configured to engage output drive members of a manipulator of a teleoperated manipulator system to which the force transmission system 210 is couplable, as would be understood by a person having ordinary skill in the art. Each of the input devices is in turn operably coupled with drive components of the force transmission system 210, such as various shafts, gears. and bearings to transmit forces from the input device to the various components of the instrument and shaft 212.

In the embodiment of FIG. 2, the force transmission system 210 includes various drive members coupled with components of the shaft and configured to transmit forces to actuate (e.g., including articulate and/or translate) actuatable components such as components of the end effector or articulable structures located more distally along the shaft in response to inputs at the input devices of the force transmission system 210, e.g., from a manual or teleoperated manipulator to which the force transmission system 210 is coupled. In addition, the force transmission system 210 can include drive members configured to provide movement to the shaft 212, such as roll motion.

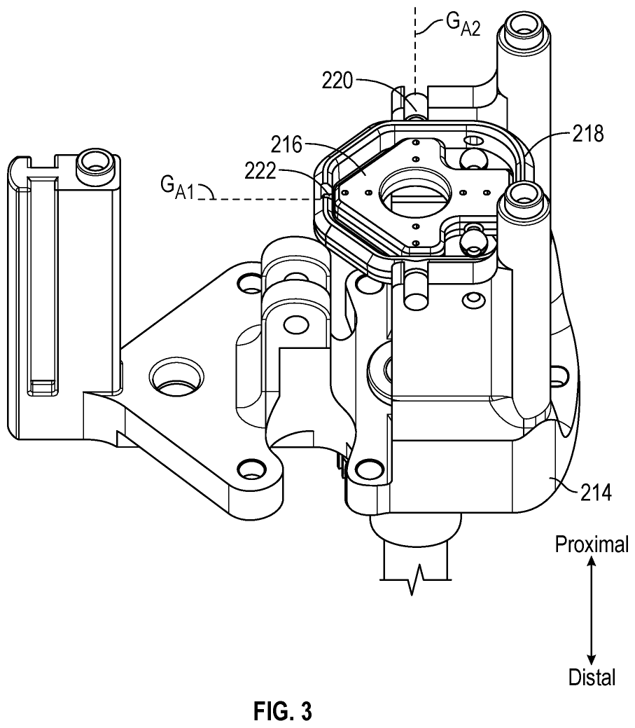

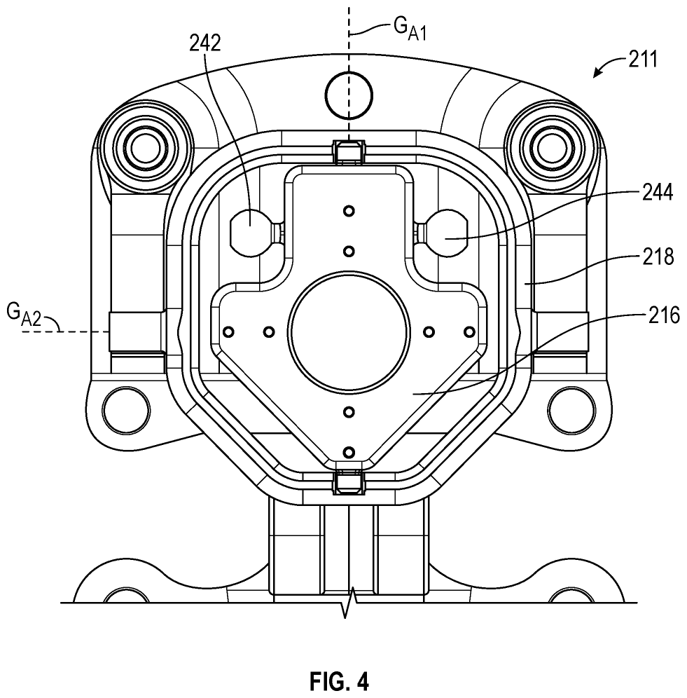

In the embodiment of FIG. 2, rotations of one or more input devices (e.g., input discs 119 shown in FIG. 1; one such disc 219 being visible in FIG. 2) are transmitted to control one or more respective degrees of freedom of the shaft 212, such as, for example, independent degrees of freedom such as pitch and yaw. The force transmission system 210 includes a gimbal assembly 211, illustrated in isolation in FIGS. 3 and 4, that receives input drive forces (such as rotations driven by a manipulator system to which the instrument and force transmission system 210 is coupled) at the input discs 219 and transmits the input drive forces to components of the shaft 212, such as an articulable structure 105 and/or end effector 104 (FIG. 1) to actuate the articulable structure 105 and/or movable components of the end effector 104 (not shown in FIG. 2). The gimbal assembly 211 includes a gimbal plate 216 pivotably coupled to a gimbal frame 218. As shown best in FIG. 3, the gimbal plate 216 is pivotably coupled to the gimbal frame 218 so as to be pivotable about a first gimbal axis G_{A1}. The gimbal frame 218 is in turn pivotably coupled to the support chassis 214 so as to be pivotable about a second gimbal axis G_{A2}. Thus, overall rotation of the gimbal plate 216 to a desired orientation about GA₁ and/or GA₂ can be effected by various combinations of rotation about GA₁, i.e., rotations of the gimbal plate 216 within the gimbal frame 218, and rotations of the gimbal plate 216 and gimbal frame 218 together about GA₂.

The first gimbal axis G_{A1} and the second gimbal axis G_{A2} can be orthogonal to one another, as shown in FIG. 2. Such an arrangement can provide substantially independent degrees of freedom of movement to actuatable components, as will be described in further detail below. In other embodiments, the first gimbal axis G_{A1} and second gimbal axis G_{A2} can be non-orthogonal to one another.

The gimbal plate 216 is pivotably coupled to the gimbal frame 218 via journal bearing surfaces 222, as best shown in FIG. 3, and the gimbal frame 218 is pivotably coupled to the support chassis 214 via journal bearing surfaces 220. Other bearing arrangements such as ball bearings, roller bearings, or other components can be used to couple the gimbal plate 216 to the gimbal frame 218 and to couple the gimbal frame 218 to the support chassis 214 without departing from the scope of the present disclosure.

The gimbal plate 216 can be operably coupled to the input discs 219 such that rotation of an input disc 119 causes corresponding rotation of the gimbal plate 216 relative to the support chassis 214 along the first gimbal axis G_{A1} (or the second gimbal axis G_{A2}). For example, the gimbal plate 216 can be mechanically coupled to one or more of the input discs 219 by a set of drive components including, for example, one or more leadscrews, lever arms, connecting rods, ball and socket joints, or other components. In some embodiments, two input discs 219 can provide the necessary input forces to move the gimbal assembly 211 in the two degrees of freedom associated with the first gimbal axis G_{A1} and the second gimbal axis G_{A2}.

Referring still to FIG. 2, the drive components that are coupled with two input discs 219 to drive the gimbal assembly 211 will now be described. The force transmission system 210 includes first and second leadscrews 224 and 226, each of which are coupled to rotate with an associated input disc 219. Each of the first and second leadscrews 224, 226 (only a portion of 226 being visible in FIG. 2) are further coupled to respective first and second lever arms 228, 230, which are pivotably coupled at a pivot axis 232 to the support chassis 214.

End portions 229 and 231 of the first and second lever arms 228, 230 engage an internally threaded collar 234 that is engaged with the respective leadscrew 224, 226. In this way, rotation of the leadscrew 224, 226 causes the associated collar 234 to travel along the leadscrew 224, 226. Due to the engagement with the collar 234, the end portions 229, 231 of the associated lever arm 228, 230 also travels along the corresponding leadscrew 224, 226, thereby causing the lever arm 228, 230 to pivot about the pivot axis 232.

Referring now to FIG. 4, the gimbal assembly 211 is shown in plan view with other components omitted for ease of illustration. As shown in FIG. 4, the gimbal plate 216 includes spherical joints 242 and 244. The spherical joints 242 and 244 are configured to be coupled to connecting rods 238, 240 as discussed above in connection with FIGS. 2 and 3. The spherical joints 242 and 244 are positioned at locations within a plane defined by the gimbal plate 216 and offset from first gimbal axis G_{A1} and second gimbal axis G_{A2}. Because the spherical joints 242 and 244 are offset from axes G_{A1} and G_{A2}, force applied to the spherical joints 242 and 244 by the connecting rods 238 and 240 as a result of rotational inputs at the input discs 219 that drive the leadscrews 224, 226 and corresponding lever arms 228, 230 (FIG. 2) creates a moment about G_{A1} and/or G_{A2} to pivot the gimbal plate 216 relative to the support chassis 214 about G_{A1} and/or G_{A2}.

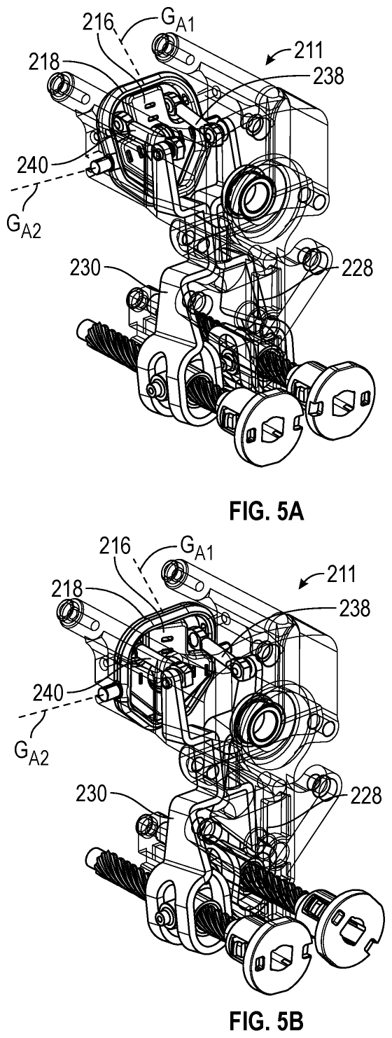

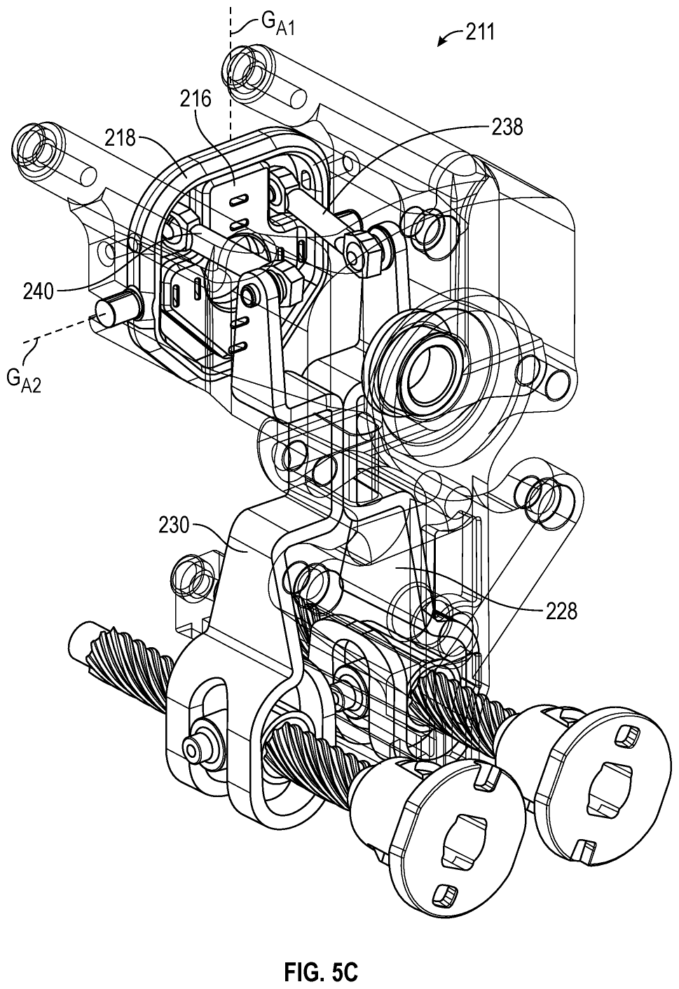

For example, referring to FIGS. 5A and 5B, actuation of the lever arms 228, 230 can impart a desired orientation to the gimbal plate 216 about axes G_{A1} and G_{A2}. In FIG. 5A, both lever arms 228, 230 are actuated in the same direction to generate rotation of the gimbal frame 218 (and gimbal plate 216 within the gimbal frame 218) about axis G_{A2}. In FIG. 5B, the lever arms 228, 230 are actuated in opposite directions to generate rotation of the gimbal plate 216 about axis G_{A1} (within a given orientation of the gimbal frame 218 about axis G_{A2}). Various combinations of movements of the first and second lever arms 228, 230 can be used to generate the desired orientation of the gimbal plate 216 and gimbal frame 218 including any desired rotations about G_{A1} and G_{A2} and combinations thereof. Referring to FIG. 5C, the lever arms 228, 230 are both in a neutral position and the gimbal plate 216 and gimbal frame 218 are thus neutrally oriented about G_{A1} and G_{A2}.

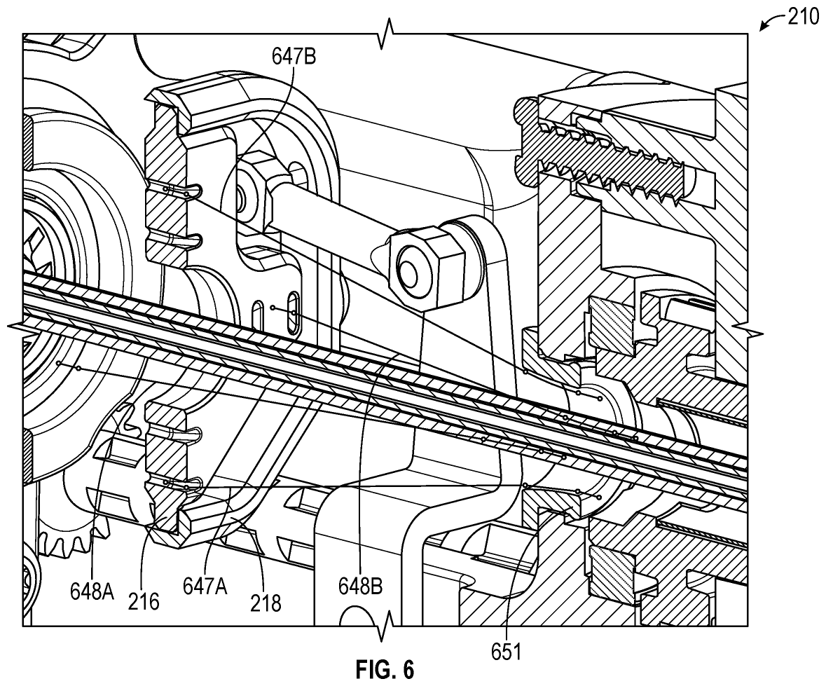

While no actuation members are shown in connection with FIGS. 2-5B for simplification of illustration, actuation members, such as cables or other tension and/or compression members, can be coupled to the gimbal plate 216 and to one or more actuatable components so as to transmit force due to motion of the gimbal plate 216 to the one or more actuatable components, as shown and discussed further below in connection with FIG. 6.

Referring now to FIG. 6, a perspective, partial cutaway drawing of the force transmission mechanism 210 is shown. Actuation members 647A, 647B are coupled to the gimbal plate 216 and extend distally from the gimbal plate 216 and through a bell mouth 651, which guides the actuation members 647A, 647B into the shaft 212. Actuation members 647A, 647B are coupled at distal end portions thereof to, for example, an articulable member coupled to the shaft 212, such as articulable member 105 (FIG. 1). The actuation members 647A, 647B intersect axis G_{A2}, about which the gimbal frame 218 rotates.

Actuation members 648A, 648B are coupled to the gimbal plate 216 and extend distally from the gimbal plate 216 and through the bell mouth 651, which guides the actuation members 648A, 648B into the shaft 212. Actuation members 648A, 648B are coupled at distal end portions thereof to, for example, an articulable member coupled to the shaft 212, such as articulable member 105 (FIG. 1). Actuation members 648A, 648B intersect axis G_{A1}, about which the gimbal plate 216 rotates within the gimbal frame 218.

In use, when the gimbal frame 218 is rotated about axis G_{A2}, i.e., as shown in FIG. 5A, actuation member 647B translates proximally and actuation member 647A translates distally, causing articulation of the articulable member 105 (see FIG. 1) in a degree of freedom (e.g., pitch or yaw) associated with actuation members 647A and 647B. Similarly, when the gimbal plate 216 is rotated within the gimbal frame 218 about axis G_{A1}, i.e., as shown in FIG. 5B, actuation member 648A translates proximally and actuation member 648B translates distally, causing articulation of the articulatable member 105 in another degree of freedom (e.g., the other of pitch and yaw) associated with actuation members 648A, 648B. As noted above in connection with FIGS. 5A and 5B, various combinations of rotations about axes G_{A1} and G_{A2} can provide any desired articulation of the articulable member 105 about the relevant degrees of freedom.

In some gimbal-type systems, due to the kinematic relationships between the actuation members and the gimbal plate, each of the actuation members may translate (i.e., move in a linear direction) in opposite directions a different amount at the articulatable structure 105 for a given amount of rotation of the gimbal plate. That is, a given translation of a first actuation member in one direction may be associated with a given translation of a second actuation member in an opposite direction by a different amount. In other words, the arrangement of the gimbal plate and the actuation members is not length conservative. In some embodiments of instruments, such as instrument 100, a given amount of articulation of the articulable structure 105 causes equivalent translational movement in opposite directions in the associated actuation members, i.e., the articulable structure 105 is length conservative. Thus, actuating the articulable structure 105 with a conventional gimbal-type mechanism can potentially result in slack in one actuation member, particularly at higher levels of rotation of the gimbal assembly from a neutral position. In extreme cases, the excessive slack can result in dislocation of the articulable structure 105.

Accordingly, in various embodiments of the present disclosure, a gimbal assembly can include various features configured to maintain substantial length conservation of the actuation members. For example, various embodiments contemplate a gimbal plate thickness that results in the actuation members having an exit location from the plate that is offset from the one or more axes about which the gimbal plate rotates. The thickness of the gimbal plate can be chosen based on various factors such as the diameter of the actuation members, the distance of each actuation member from the associated axis about which the gimbal plate rotates to actuate the actuation member, the dimension from the gimbal assembly to the bell mouth along a longitudinal axis of the instrument, or other kinematic characteristics of the gimbal assembly. Tailoring the thickness of the plate in this manner can modify the kinematics of the gimbal assembly to reduce or eliminate slack development in the actuation members, maintain substantial length conservation, and correspondingly reduce the potential for dislocation of the articulable structure controlled by the actuation members and gimbal assembly.

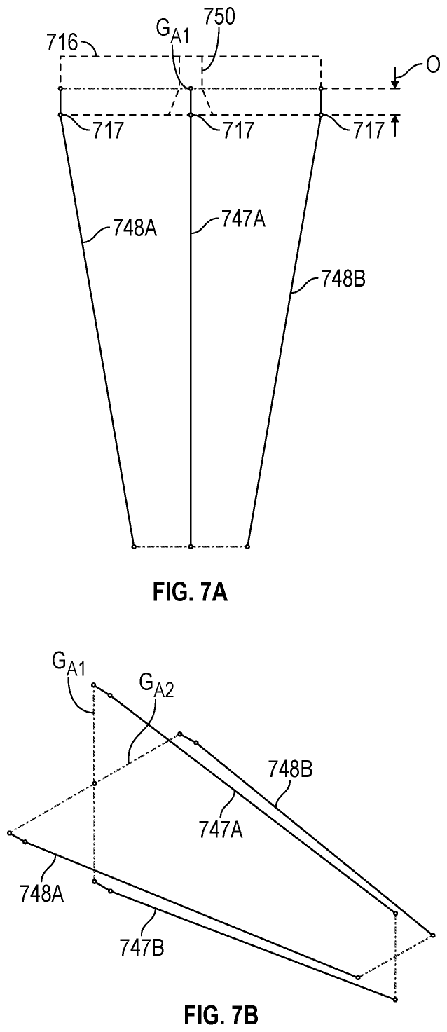

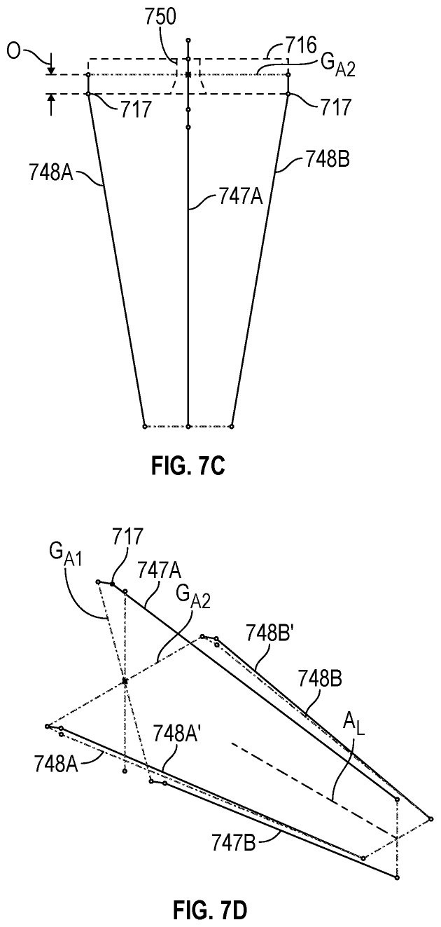

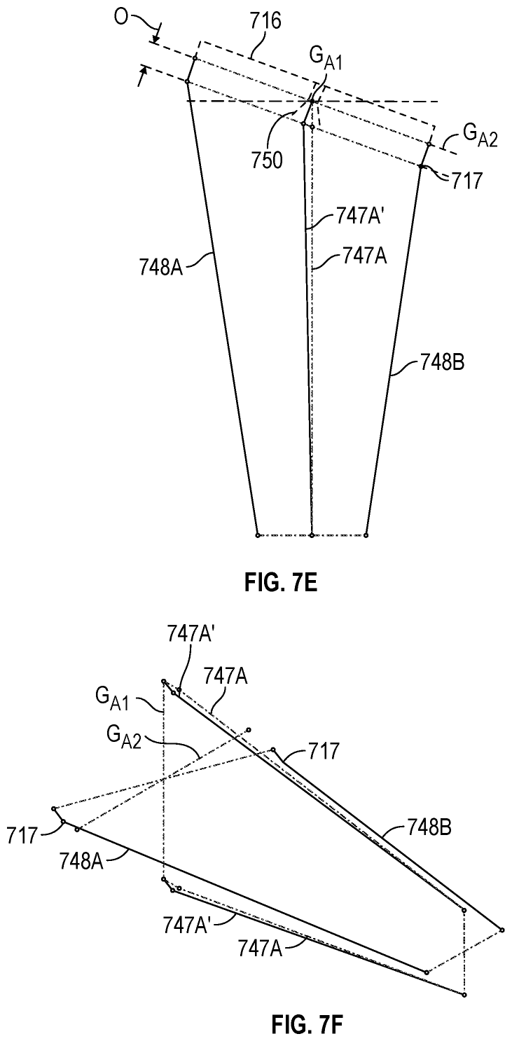

Referring to FIGS. 7A through 7F, various schematic side and perspective views of actuation members 647A, 647B, 648A, and 648B are shown in various articulated positions with other portions of the assembly, such as the gimbal plate and gimbal frame, omitted to more clearly show the geometry of the actuation members. Referring to FIGS. 7A and 7B, the gimbal plate 716 (FIG. 7A) has a thickness that defines an offset O from the location 717 actuation member 747A breaks over the gimbal plate 716 to a plane in which rotational axes G_{A1} and G_{A2} lie. Upon rotation of the gimbal plate 716 about axis G_{A2}, as shown in FIGS. 7C and 7D, due to the offset O, actuation members 747A and 747B break over the gimbal plate 716 at the location 717 on actuation member 747A. Due to the offset O, the actuation member 747B is thus positioned closer to a central axis A_{L} of the instrument (e.g., instrument 100 in FIG. 1) relative to a path that would be followed by the actuation member 747B in the absence of the offset O. The distance the actuation member 747B is positioned closer to the central axis AL is a function of various factors including the offset O, the angle the gimbal plate 716 has rotated about G_{A2}, and a distance D along the gimbal plate 716 from the axis G_{A2} to the actuation member 747B. Similarly, the actuation member 747A is positioned farther from the central axis A_{L} of the instrument relative to a path that would be followed by the actuation member 747A in the absence of the offset O. The inward direction of actuation member 747B toward the central axis A_{L} at the location 717 as a result of the offset O can reduce excess slack generated in actuation member 747B relative to the uptake of actuation member 747A and thereby improve the length conservation characteristics of the gimbal assembly and reduce the likelihood of dislocation of the articulable structure 105 due to excess slack.

The thickness of the gimbal plate 716 and creation of the offset O of the location 717 in the embodiment described with relation to FIGS. 7A-7D provides kinematic characteristics that permit substantial length conservation of the actuation members 747A and 747B. Similar features and benefits can be associated with actuation members 748A and 748B, although the discussion above focuses on members 747A and 747B for brevity.

As discussed above, the thickness of the gimbal plate and associated offset of the exit location of a pair of actuation members can maintain substantial length conservation in that pair of actuation members. However, undesired lateral movement of those actuation members can occur when the gimbal plate is pivoted about another axis to actuate a different pair of actuation members. With reference to FIGS. 7C and 7D, when the gimbal plate 716 is rotated about G_{A2}, the offset O could cause undesired lateral movement of the actuation members 748A and 748B as the gimbal plate rotates about axis G_{A2}. For example, with continued reference to FIGS. 7C and 7D, solid lines 748A′ and 748B′ show such a lateral movement of actuation members 748A and 748B resulting from rotation of the gimbal plate about axis G_{A2} due to the offset O. Similarly, with reference to FIGS. 7E and 7F, in which the actuation members are shown as if the gimbal plate is rotated about axis G_{A1}, solid lines 747A′ and 747B′ show lateral movement in actuation members 747A and 747B resulting from rotation of the gimbal plate about axis G_{A2}.

In some embodiments herein, the gimbal plate 716 can include additional features to mitigate (e.g., reduce or eliminate) lateral movement of non-actuated actuation members (i.e., actuation members positioned along a given axis as the gimbal plate is rotated about that given axis). For example, referring to FIGS. 7C-7F the gimbal plate 716 can include various features, such as shaped apertures, that reduce or eliminate lateral movement of actuation members aligned with an axis about which the gimbal plate is rotated. Such apertures can be configured so that the actuation members aligned with the rotational axis does not “see” the thickness of the gimbal plate and the offset O, while the actuation members actuated by rotation of that rotational axis “see” the thickness and offset O and accordingly experience improved length conservation as discussed above.

Referring to FIG. 7E, one example of an aperture 750 (shown in dashed lines) according to the present disclosure is shown. Aperture 750 that has a shape in at least the distal face of the gimbal plate that is elongated in the direction of axis G_{A2}. The elongated shape of the aperture 750 can comprise an inverted “Y” shape, with the center of the Y being aligned with G_{A1}. Thus, as seen in FIG. 7E, the actuation member 747A is free to move within the aperture and follows the path indicated by dashed lines. In contrast, in the absence of Y-shaped aperture 750, actuation member 747A would follow path indicated by solid line 747A′ (i.e., exhibiting lateral movement based on rotation of the gimbal plate about axis G_{A1}). Stated another way, the aperture 750 is shaped to enable actuation member 747A to remain at a neutral location regardless of rotation of the gimbal plate 716 about G_{A1}. Thus, the actuation member 747A does not “swing” about G_{A1} in the manner indicated by path 747A′, and rotation of the gimbal plate 716 about G_{A1} does not introduce unwanted lateral movement in actuation member 746. Actuation members 748A, 748B can be provided in apertures having a similar configuration as aperture 750 but elongated along G_{A1} such that rotation of the gimbal plate 716 about G_{A2} does not cause undesirable lateral movement of actuation members 748A, 748B. Thus, the actuation members 748A, 748B break over the gimbal plate 716 at a location offset by O from axes G_{A1} and G_{A2} when the gimbal plate 716 is rotated about G_{A1}, and the actuation members 748A, 748B break over the gimbal plate 716 at a longitudinally different location (i.e., within the elongated apertures 750) when the gimbal plate is rotated about G_{A2}. Likewise, actuation members 747A and 747B break over the gimbal plate 716 at a location offset by O from axis G_{A2} upon rotation of the gimbal plate about G_{A2} and break over the gimbal plate within the elongated apertures upon rotation of the gimbal plate 716 about G_{A1}.

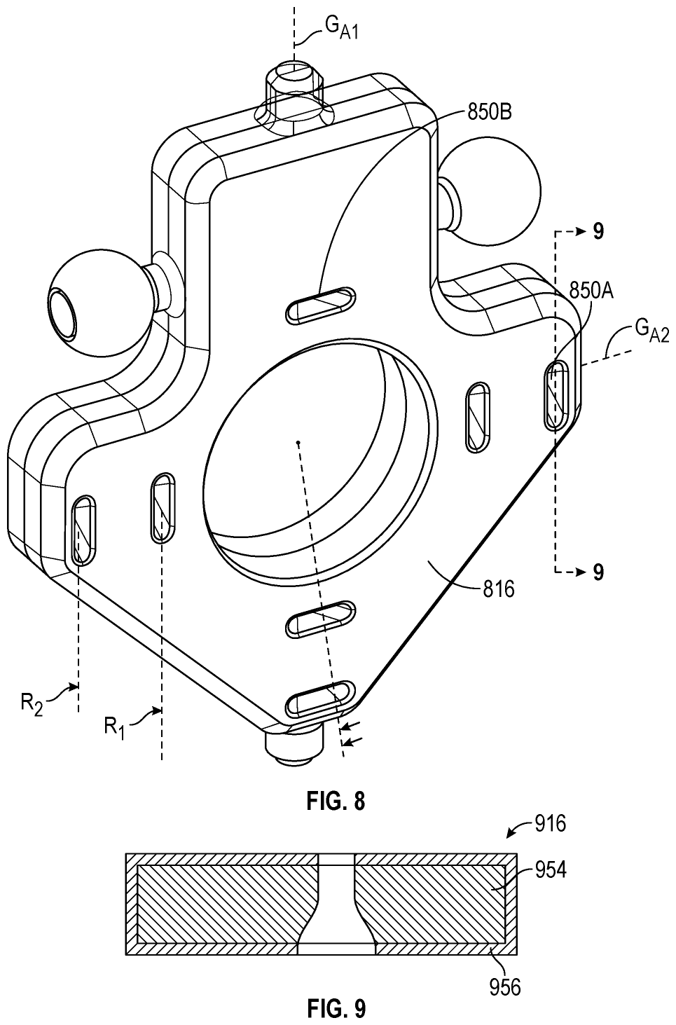

FIG. 8 shows an embodiment of a gimbal plate similar to that described in connection with FIGS. 2-5B and including the thickness and elongated apertures discussed in connection with FIGS. 7A-7E. Gimbal plate 816 includes apertures 850A elongated in a direction parallel to G_{A1} and apertures 850B elongated in a direction parallel to G_{A2}. Each of the apertures 850A and 850B (which correspond to apertures 750 indicated by dashed lines in FIGS. 7A, 7C, and 7E) have a generally inverted Y shape, with the elongated portion of each aperture converging to a circular cross section within the gimbal plate 816, e.g., aligned with an imaginary plane within the gimbal plate 816 within which G_{A1} and G_{A2} lie. The apertures 850A and 850B can also be described as having a tapered cross-sectional shape.

The gimbal plate 816 also includes features configured to facilitate modularity of design and permit the gimbal assembly 211 (FIG. 2) to be used with various instruments having different characteristics. For example, as shown in FIG. 8, the gimbal plate 816 includes apertures positioned on a first circle having a first radius R1 and a second circle having a second radius R2. The second radius R2 is larger than the first radius R1. The gimbal plate 816 can be used with different instruments with different shaft diameters by using the apertures positioned on the first circle, e.g., for instruments with a relatively smaller shaft diameter and using the apertures positioned on the second circle, e.g., for instruments with a relatively larger shaft diameter. In this way, the same components can be used across a range of instruments to contribute to manufacturing efficiencies, fewer inventory parts, etc. Additionally, the differing aperture positioning can facilitate use of the gimbal assembly 211 with different types of actuation members. For example, coupling the actuation members at the apertures positioned at the second circle can provide a relatively greater stroke (i.e., translational movement) for a given angular rotation of the gimbal plate to facilitate use of actuation members having relatively low stiffness, such as stranded cables or tendons. The size of the first circle and/or second circle can be determined based on factors such as the diameter of the instrument shaft (e.g., shaft 112 shown in FIG. 1), material and construction of actuation members, and kinematic characteristics of the articulable structure 105 (FIG. 1), and other factors.

The gimbal plate and gimbal frame discussed in connection with various embodiments herein can be made from relatively high strength material such as stainless steel, or other metal or metal alloy. Alternatively or additionally, the gimbal plate and/or gimbal frame can comprise a composite material, a composite structure comprising multiple materials, or combinations thereof. In the embodiment of FIG. 9, which shows a cross-sectional view of a gimbal plate 916, the gimbal plate 916 comprises a polymer core portion 954 and a metal shell portion 956. The polymer core portion 954 provides thickness to the gimbal plate 916 as discussed above, while the metal shell portion 956 provides high strength at locations at which the actuation members contact the gimbal plate 916 so that localized forces due to the tension in the actuation members does not damage the gimbal plate 916. Other materials, structures, and combinations of materials for the gimbal plate 916 are considered within the scope of the present disclosure. Further, while the gimbal assembly 211 (FIG. 2) discussed herein is shown with an exemplary four actuation members, each pair of which operates a degree of freedom of a two-degree of freedom articulable structure 105 (FIG. 1), fewer actuation members or more actuation members associated with fewer or more degrees of freedom are within the scope of the disclosure. Further, the gimbal assembly 211 is not limited to actuating articulable joint 105 and can be configured to operate various other components of instruments such as end effector components or other devices.

The gimbal assembly 211 (FIG. 2) can be configured to avoid kinematic singularities within the range of motion of the gimbal assembly 211 and associated devices, such as the articulable structure(s) 105 (FIG. 1) or end effector 104 (FIG. 1). That is, the gimbal assembly 211 can be configured such that anywhere within its range of motion, a force applied to the gimbal assembly 211 (e.g., via inputs at input devices 119, FIG. 1) generates a corresponding movement at the articulatable structure 105 or end effector 104. Further, in embodiments in which the gimbal assembly 211 is back-drivable, a force applied at the articulable structure 105 or end effector 104 anywhere within their respective ranges of motion generates corresponding movements in the gimbal assembly 211.

Instruments having force transmission systems including gimbal assemblies according to the present disclosure can facilitate lower overall cost of the instrument as a result of simplified assembly, lower part count, and modularity of parts, along with other advantages provided by the gimbal assemblies. Force transmission mechanisms according to the disclosure can include additional features configured to facilitate manufacturing and lower the overall cost of the instruments. For example, force transmission mechanisms can include features configured to reduce overall instrument part count, reduce complexity, and facilitate assembly, such as by facilitating automated assembly.

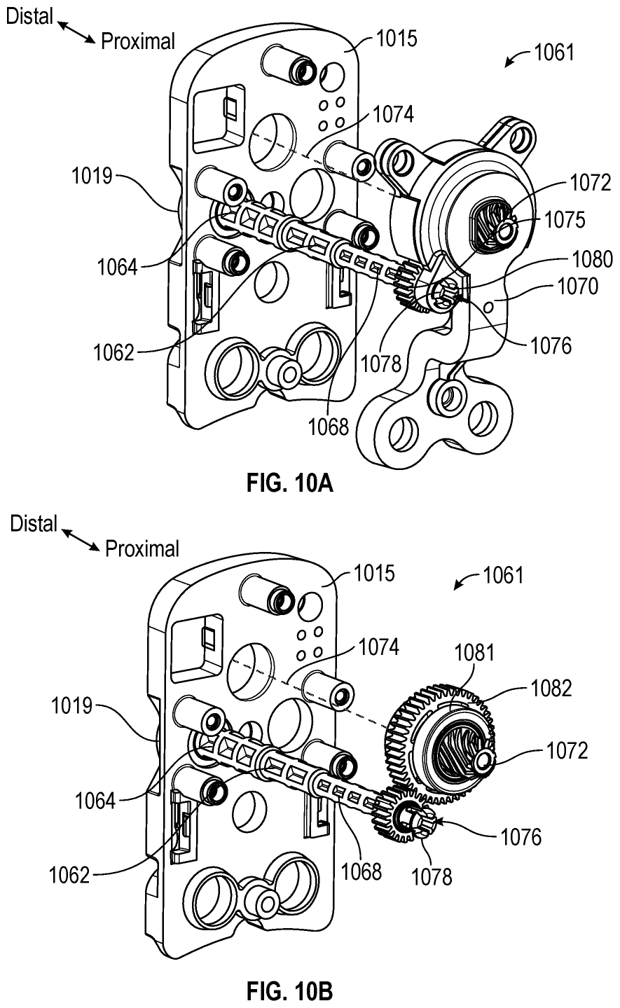

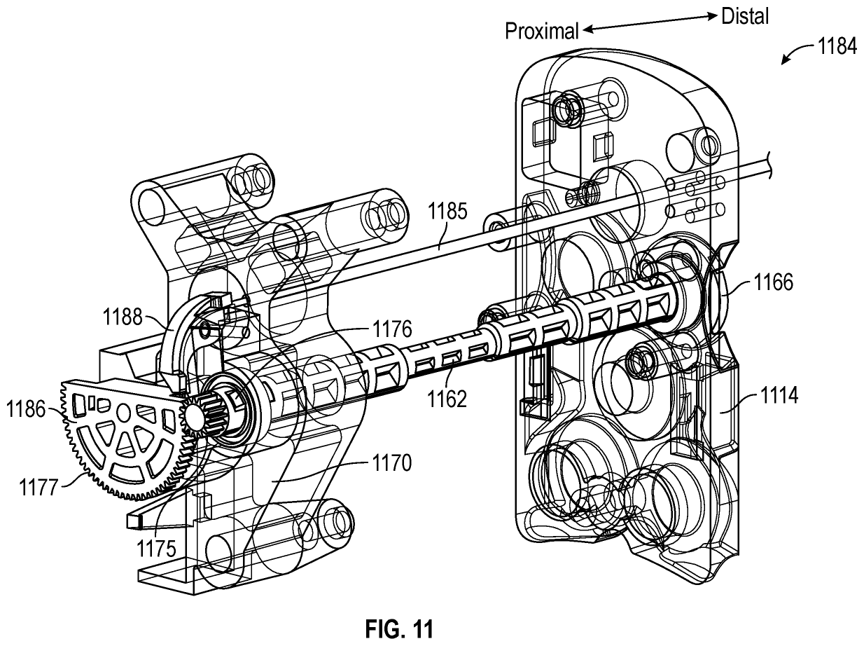

According to some embodiments of the disclosure, the force transmission system (such as force transmission system 110 shown in FIG. 1) can include various subassemblies configured to actuate functions of the end effector 104 (FIG. 1), such as actuating gripping mechanisms and/or cutting mechanisms. Such subassemblies can include features configured to simplify manufacturing by reducing overall part count and facilitating automated assembly. In addition, the components and subassemblies shown and described in connection with FIGS. 10A, 10B, and 11 can be used in conjunction with the gimbal assemblies shown and described in connection with FIGS. 2-9 above, these embodiments and subassemblies can be used alone, in combination with one another, and in combination with other gimbal and non-gimbal type systems for articulating joints.

Referring now to FIGS. 10A and 10B, and embodiment that can be used as force transmission system 210 (FIG. 1) is shown, with other components such as a gimbal assembly like that described above with reference to the embodiments of FIGS. 2-9, hidden to facilitate illustration of an end effector drive assembly 1061. The end effector drive assembly 1061 includes a driveshaft 1062, a first end portion 1064 of which can include an input device 1019 (such as corresponding to an input disc 119 shown in FIG. 1) rotatably supported by the base 1015. A second end portion 1068 of the driveshaft 1062 is rotatably supported by a driven gear carrier 1070 (the outer housing of which is shown in FIG. 10A and the gear components of which are shown in FIG. 10B with the outer housing removed) that couples ultimately to the base 1015 via other structures (such as the gimbal assembly 211 shown in FIG. 2 or any other suitable mechanisms to secure the driven gear carrier 1070 to the base 1015) not illustrated in FIG. 10A. The driven gear carrier 1070 includes a leadscrew 1072 that can be operably coupled to an actuation member 1074 that extends through the instrument shaft (e.g., shaft 112 shown in FIG. 1) to the end effector to operate an end effector component, such as a translating cutting element, a grip mechanism, or other end effector component. The actuation member 1074 can comprise a cable, a rod, or other structure configured to translate based on force applied by movement of the leadscrew 1072. In the embodiment of FIGS. 10A and 10B, the actuation member 1074 is a push/pull actuation member. That is, the actuation member 1074 can transmit both tensile and compressive forces to a component of the end effector, such as a cutting element configured to translate longitudinally, jaw members configured to open or close, or other end effector components as those having ordinary skill in the art are familiar with. However, the end effector drive assembly 1061 can be configured for use with pull-type only actuation members or other types of actuation members without limitation.

The second end portion 1068 of the driveshaft 1062 includes a pinion gear 1075 and a retention feature 1076 configured to couple the driveshaft 1062 to the driven gear carrier 1070. The retention feature 1076 can optionally be configured for tool-less assembly and may comprise one or more deflectable members 1078 configured to impart a “snap-fit” type interface with the driven gear carrier 1070. For example, the one or more deflectable members 1078 can be configured to deflect inward as the retention feature 1076 is inserted through a corresponding retention feature in the driven gear carrier 1070, such as a bore 1080 of the driven gear carrier 1070, and once the retention feature 1076 is fully inserted, the one or more deflectable members 1078 return outward from the inwardly deflected position to retain the driveshaft 1062 within the driven gear carrier 1070. In some embodiments, the one or more deflectable members 1078 can form an integral part of the driveshaft 1062. As one example, the one or more deflectable members 1078 can be co-molded with the driveshaft 1062 from a polymer material.

Referring to FIG. 10B, the driven gear carrier 1070 (FIG. 10A) is hidden to better show components coupled to and associated with the driven gear carrier 1070. For example, the driven gear carrier 1070 comprises one or more bearings 1081 that rotatably support a driven gear 1082 that is configured to be meshed with the pinion gear 1075. The driven gear 1082 comprises internal threads (not shown) configured to mesh with the threads of the leadscrew 1072. On rotation of the driveshaft 1062 and pinion gear 1075, in response to input at the input device 1019, the driven gear 1082 in turn rotates and causes translation of the leadscrew 1072 and the actuation member 1074 coupled thereto, which translational movement is transmitted to actuate the component of the end effector to which the actuation member 1074 is coupled.

Optionally, the driven gear 1082, the one or more bearings 1081, and the leadscrew 1072 can be assembled with the driven gear carrier 1070 prior to overall assembly of the force transmission system 210. Thus, rather than individually assembling the bearings 1081, driven gear 1082, leadscrew 1072, and other components individually on the force transmission system, the driven gear carrier 1070 and associated components can be provided as a completed driven gear assembly for coupling to the support chassis 214 and the driveshaft 1062, e.g., by simply pressing the driven gear carrier 1070 over the retention feature 1076 of the driveshaft 1062 and affixing (e.g., with hardware or integral coupling features) the driven gear carrier 1070 to the base chassis 1014 and/or other portions of the force transmission system 210. This arrangement can simplify assembly of the force transmission system 210 and provide additional flexibility in terms of manufacturing, because the parts associated with the driven gear carrier 1070 can be assembled prior to overall assembly of the force transmission system 210, and the retention feature 1076 of the driveshaft 1062 further reduces the part count, as it requires no separate fasteners, and simplifies assembly, since it requires no specialized tools for assembling the driveshaft 1062 and driven gear carrier 1070.

Referring now to FIG. 11, another embodiment of an end effector drive assembly 1184 of a force transmission system such as force transmission system 210 (FIG. 2) is shown. The end effector drive assembly 1184 can be used together with, or separate from, the end effector drive assembly 1061 and/or gimbal assemblies disclosed herein. The end effector drive assembly 1184 is configured for use with a pull-pull actuation member 1185, such as a cable or other flexible member, which may be used for actuation of end effector components such as to move jaws of an end effector to open and close, sometimes referred to as grip drive mechanisms as those of ordinary skill in the art are familiar, drive translation of a cutting element, or actuate another component of an end effector. Like the end effector drive assembly 1061, the end effector drive assembly 1184 can be configured for use with push/pull actuation members or pull-pull actuation elements without limitation. The end effector drive assembly 1184 includes a driveshaft 1162, similar to driveshaft 1062 described above, that is operatively coupled to an input device 1166 (for example, corresponding to input disc 119 shown in FIG. 1) at one end portion and to a pinion gear 1175 at the other, opposite end portion of the driveshaft 1162. The pinion gear 1175 is configured to be meshed with external gear teeth 1177 of a bellcrank 1186. The actuation member 1185 is coupled to the bellcrank 1186 and routed through a guide 1188 that redirects the actuation member 1185 from wrapping at least partially around the bellcrank 1186 to extending along the shaft 112 (FIG. 1) of the instrument. Upon rotation of the driveshaft 1162, e.g., as a result of a rotational input at input device 1166, the bellcrank rotates and tensions the actuation member 1185, thereby causing the actuation member 1185 to close jaws of the end effector 104 (FIG. 1), actuate a cutting element, or cause other movement or actuation of the end effector such as any component driven by translation of the actuation member 1185

The bellcrank 1186, guide 1188, and one or more bearings 1181 are coupled to a driven gear carrier 1170, which is in turn fastened to the base chassis 1114, e.g., via coupling to a gimbal assembly (not shown for clarity). As discussed above, these components can be fastened together prior to overall force transmission system assembly to facilitate manufacturing flexibility. In some embodiments, the driveshaft 1162 can include one or more retention features 1176 configured to facilitate tool-less coupling of the driveshaft 1162 to the driven gear carrier 1170, in a manner similar to that discussed above in connection with FIGS. 10A and 10B.

In various embodiments, it is contemplated that the force transmission systems with drive assemblies 1061 and/or 1184, optionally in conjunction with the gimbal assemblies discussed herein, can be used with, for example, instruments having the various configurations recited in U.S. Provisional Patent Application 63/279,500 (filed Nov. 15, 2021) and titled “INSTRUMENT END EFFECTOR WITH JAW MECHANISM AND MOVEABLE COMPONENT AND RELATED DEVICES, SYSTEMS AND METHODS,” the entire contents of which are incorporated by reference herein.

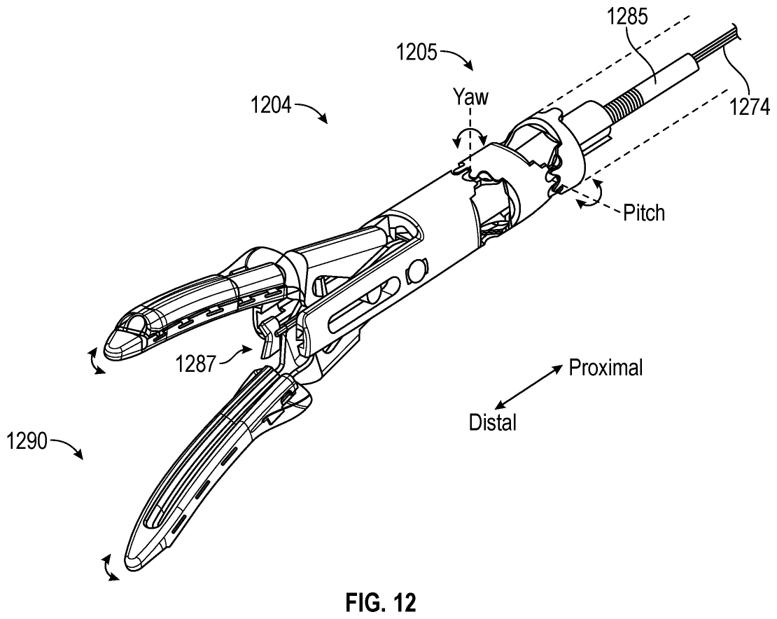

FIG. 12 shows a perspective view of an end effector 1204 according to one embodiment of the present disclosure for which the force transmission systems and drive assemblies described herein may be operably coupled to actuate the end effector. The end effector 1204 comprises a jaw mechanism 1290 and a cutting element 1287. The jaw mechanism 1290 and the cutting element 1287 can be actuated via end effector drive assemblies 1061 and 1184 as discussed herein in connection with FIGS. 10A, 10B and 11. For example, in the embodiment of FIG. 12, the jaw mechanism 1290 can be operably coupled to end effector drive assembly 1061 via actuation member 1285, and the cutting element 1287 can be operably coupled to the end effector drive assembly 1061 via actuation member 1274. Other arrangements, such as the cutting element being operably coupled to the end effector drive assembly 1184 and the jaw mechanism being operably coupled to the end effector drive assembly 1061, and/or other arrangements and combinations thereof are within the scope of the disclosure.

Various embodiments of the present disclosure provide force transmission systems that facilitate modularity, automated assembly, and adaptability to various applications. Further, they can facilitate overall ease of manufacturing, assembly, and contribute to reliability of the device.

Embodiments described herein may be used, for example, with remotely operated, computer-assisted systems (such, for example, teleoperated surgical systems) such as those described in, for example, U.S. Pat. No. 9,358,074 (filed May 31, 2013) to Schena et al., entitled “Multi-Port Surgical Robotic System Architecture”. U.S. Pat. No. 9,295,524 (filed May 31, 2013) to Schena et al., entitled “Redundant Axis and Degree of Freedom for Hardware-Constrained Remote Center Robotic Manipulator”, and U.S. Pat. No. 8,852,208 (filed Aug. 12, 2010) to Gomez et al., entitled “Surgical System Instrument Mounting”, each of which is hereby incorporated by reference in its entirety. Further, embodiments described herein may be used, for example, with various da Vinci® Surgical Systems, commercialized by Intuitive Surgical, Inc., of Sunnyvale, California.

The embodiments described herein are not limited to the surgical systems noted above, and various other teleoperated, computer-assisted surgical system configurations may be used with the embodiments described herein. Further, although various embodiments described herein are discussed in connection with a manipulating system of a teleoperated surgical system, the present disclosure is not limited to use with a teleoperated surgical system. Various embodiments described herein can optionally be used in conjunction with hand-held, manual instruments.

As discussed above, in accordance with various embodiments, force transmission systems of the present disclosure are configured for use in teleoperated, computer-assisted surgical systems employing robotic technology (sometimes referred to as robotic surgical systems). Referring now to FIG. 13, an embodiment of a manipulator system 1000 of a computer-assisted surgical system, to which surgical instruments are configured to be mounted for use, is shown. Such a surgical system may further include a user control system, such as a surgeon console (not shown) for receiving input from a user to control instruments coupled to the manipulator system 1000, as well as an auxiliary system, such as auxiliary systems associated with the da Vinci® systems noted above.

As shown in the embodiment of FIG. 13, a manipulator system 1000 includes a base 1020, a main column 1040, and a main boom 1060 connected to main column 1040. Manipulator system 1000 also includes a plurality of manipulator arms 1010, 1011, 1012, 1013, which are each connected to main boom 1060. Manipulator arms 1010, 1011, 1012, 1013 each include an instrument mount portion 1022 to which an instrument 1030 may be mounted, which is illustrated as being attached to manipulator arm 1010. While the manipulator system 1000 of FIG. 13 is shown and described having a main boom 1060 to which the plurality of manipulator arms are coupled and supported thereby, in other embodiments, the plurality of manipulator arms can be coupled and supported by other structures, such as an operating table, a ceiling, wall, or floor of an operating room, etc.

Instrument mount portion 1022 comprises a drive output assembly 1023 and a cannula mount 1024, with a transmission mechanism 1034 (which may generally correspond to the force transmission system 110 discussed in connection with FIG. 1 or 210 discussed in connection with FIG. 2) of the instrument 1030 connecting with the drive output assembly 1023, according to an embodiment. Cannula mount 1024 is configured to hold a cannula 1036 through which a shaft 1032 of instrument 1030 may extend to a surgery site during a surgical procedure. Drive output assembly 1023 contains a variety of drive and other mechanisms that are controlled to respond to input commands at the surgeon console and transmit forces to the transmission mechanism 1034 to actuate the instrument 1030. Although the embodiment of FIG. 13 shows an instrument 1030 attached to only manipulator arm 1010 for ease of viewing, an instrument may be attached to any and each of manipulator arms 1010, 1011, 1012, 1013.

Other configurations of surgical systems, such as surgical systems configured for single-port surgery, are also contemplated. For example, with reference now to FIG. 14, a portion of an embodiment of a manipulator arm 2140 of a manipulator system with two surgical instruments 2309, 2310 in an installed position is shown. The surgical instruments 2309, 2310 can generally correspond to instruments discussed above, such as instrument 100 disclosed in connection with FIG. 1. The schematic illustration of FIG. 14 depicts only two surgical instruments for simplicity, but more than two surgical instruments may be mounted in an installed position at a manipulator system as those having ordinary skill in the art are familiar with. Each surgical instrument 2309, 2310 includes a shaft 2320, 2330 that at a distal end has a moveable end effector or an endoscope, camera, or other sensing device, and may or may not include a wrist mechanism (not shown) to control the movement of the distal end.

In the embodiment of FIG. 14, the distal end portions of the surgical instruments 2309, 2310 are received through a single port structure 2380 to be introduced into the patient. As shown, the port structure includes a cannula and an instrument entry guide inserted into the cannula. Individual instruments are inserted into the entry guide to reach a surgical site.

Other configurations of manipulator systems that can be used in conjunction with the present disclosure can use several individual manipulator arms. In addition, individual manipulator arms may include a single instrument or a plurality of instruments. Further, as discussed above, an instrument may be a surgical instrument with an end effector or may be a camera instrument or other sensing instrument utilized during a surgical procedure to provide information, (e.g., visualization, electrophysiological activity, pressure, fluid flow, and/or other sensed data) of a remote surgical site.

Transmission systems 2385, 2390 (which may generally correspond to force transmission system 110 disclosed in connection with FIG. 1) are disposed at a proximal end of each shaft 2320, 2330 and connect through a sterile adaptor 2400, 2410 with drive assemblies 2420, 2430. Drive assemblies 2420, 2430 contain a variety of internal mechanisms (not shown) that are controlled by a controller (e.g., at a control cart of a surgical system) to respond to input commands at a surgeon side console of a surgical system to transmit forces to the transmission mechanisms 2385, 2390 to actuate surgical instruments 2309, 2310.

The embodiments described herein are not limited to the embodiments of FIG. 13 and FIG. 14, and various other teleoperated, computer-assisted surgical system configurations may be used with the embodiments described herein. The diameter or diameters of an instrument shaft and end effector are generally selected according to the size of the cannula with which the instrument will be used and depending on the surgical procedures being performed.

This description and the accompanying drawings that illustrate various embodiments should not be taken as limiting. Various mechanical, compositional, structural, electrical, and operational changes may be made without departing from the scope of this description and the invention as claimed, including equivalents. In some instances, well-known structures and techniques have not been shown or described in detail so as not to obscure the disclosure. Like numbers in two or more figures represent the same or similar elements. Furthermore, elements and their associated features that are described in detail with reference to one embodiment may, whenever practical, be included in other embodiments in which they are not specifically shown or described. For example, if an element is described in detail with reference to one embodiment and is not described with reference to another embodiment, the element may nevertheless be claimed as included in the other embodiment.

For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Further, this description's terminology is not intended to limit the invention. For example, spatially relative terms—such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly.

Further modifications and alternative embodiments will be apparent to those of ordinary skill in the art in view of the disclosure herein. For example, the devices and methods may include additional components or steps that were omitted from the diagrams and description for clarity of operation. Accordingly, this description is to be construed as illustrative only and is for the purpose of teaching those skilled in the art the general manner of carrying out the present teachings. It is to be understood that the various embodiments shown and described herein are to be taken as exemplary. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. Changes may be made in the elements described herein without departing from the spirit and scope of the present teachings and following claims.

It is to be understood that the particular examples and embodiments set forth herein are non-limiting, and modifications to structure, dimensions, materials, and methodologies may be made without departing from the scope of the present teachings.

Other embodiments in accordance with the present disclosure will be apparent to those skilled in the art from consideration of the specification and practice of the invention disclosed herein. It is intended that the specification and examples be considered as exemplary only, with the following claims being entitled to their fullest breadth, including equivalents, under the applicable law.

1. An instrument, comprising:
a shaft comprising a distal end portion, a proximal end portion, and a longitudinal axis extending between the distal end portion and the proximal end portion;
a moveable component coupled to the distal end portion of the shaft;
a drive assembly coupled to the proximal end portion of the shaft;
a first actuation member extending from the drive assembly along the shaft and coupled to the moveable component; and
a second actuation member extending from the drive assembly along the shaft and coupled to the moveable component,
wherein the drive assembly comprises a gimbal assembly rotatable about a first axis and a second axis, the gimbal assembly comprising a first aperture and a second aperture;
wherein the first actuation member is routed through the first aperture and coupled to the gimbal assembly, the first actuation member being longitudinally translatable relative to the shaft in response to rotation of the gimbal assembly about the first axis;
wherein the second actuation member is routed through the second aperture and coupled to the gimbal assembly, the second actuation member being longitudinally translatable relative to the shaft in response to rotation of the gimbal assembly about the second axis;
wherein the first aperture defines an elongated opening in a direction perpendicular to the second axis, and
wherein the second aperture defines an elongated opening in a direction perpendicular to the first axis.

2. The instrument of claim 1, wherein the first aperture comprises a tapered cross section in a plane perpendicular to the second axis, and the second aperture comprises a tapered cross section in a plane perpendicular to the first axis.

3. The instrument of claim 1, wherein the first axis and second axis are perpendicular to one another.

4. The instrument of claim 1, wherein:
in a first position of the gimbal assembly rotated about the second axis from a neutral position, the first actuation member exits from the gimbal assembly at a first longitudinal location along the first actuation member; and
in a second position of the gimbal assembly rotated about the first axis from the neutral position, the first actuation member exits from the gimbal assembly at a second longitudinal location different from the first longitudinal location.

5. The instrument of claim 4, wherein the second longitudinal location is proximal of the first longitudinal location.

6. The instrument of claim 1, wherein:
in a first position of the gimbal assembly rotated about the first axis from a neutral position, the second actuation member exits from the gimbal assembly at a first longitudinal location along the second actuation member; and
in a second position of the gimbal assembly rotated about the second axis from the neutral position, the second actuation member exits from the gimbal assembly at a second longitudinal location different from the first longitudinal location.

7. The instrument of claim 6, wherein the second longitudinal location is proximal of the first longitudinal location.

8. The instrument of claim 1, wherein the moveable component comprises an articulable structure articulable in pitch and yaw motions.

9. The instrument of claim 1, wherein:
the moveable component is a wrist mechanism coupling an end effector to the distal end portion of the shaft.

10. The instrument of claim 1, wherein the gimbal assembly comprises:
a gimbal frame rotatable relative to the shaft about the second axis; and
a gimbal plate rotatable relative to the gimbal frame about the first axis.

11. The instrument of claim 1, wherein the gimbal assembly comprises a composite structure comprising a polymer core portion and a metal shell portion at least partially surrounding the polymer core portion.

12. An instrument comprising:
a shaft comprising a distal end portion, a proximal end portion, and a longitudinal axis extending between the distal end portion and the proximal end portion;
a moveable component coupled to the distal end portion of the shaft;
a drive assembly coupled to the proximal end portion of the shaft, the drive assembly comprising a gimbal assembly rotatable about a first axis and a second axis;
a first actuation member coupled to the gimbal assembly and extending from the drive assembly along the shaft and coupled to the moveable component; and
a second actuation member coupled to the gimbal assembly and extending along the drive assembly along the shaft and coupled to the moveable component,
wherein:
in a first position of the gimbal assembly rotated about the first axis from a neutral position, the first actuation member exits from the gimbal assembly at a first longitudinal location along a length of the first actuation member,
in a second position of the gimbal assembly rotated about the second axis from the neutral position, the first actuation member exits from the gimbal assembly at a second longitudinal location along the length of the first actuation member
the first longitudinal location along the length of the first actuation member is distal of the second longitudinal location along the length of the first actuation member,
in a third position of the gimbal assembly rotated about the second axis from a neutral position, the second actuation member exits from the gimbal assembly at a third longitudinal location along a length of the second actuation member; and
in a fourth position of the gimbal assembly rotated about the first axis from a neutral position, the second actuation member exits from the gimbal assembly at a fourth longitudinal location along the length of the second actuation member, and
the third longitudinal location along the length of the second actuation member is distal of the fourth longitudinal location along the length of the second actuation member.

13.-16. (canceled)

17. The instrument of claim 13, wherein:
the gimbal assembly comprises a first aperture having at least a portion elongated in a direction parallel to the first axis and a second aperture having at least a portion elongated in a direction parallel to the second axis; and
at least a portion of the first actuation member is coupled to the gimbal assembly at the first aperture and extends through the first aperture and at least a portion of the second actuation member is coupled to the gimbal assembly at the second aperture and extends through the second aperture.

18. An instrument, comprising:
a shaft comprising a proximal end portion and a distal end portion;
a moveable component coupled to the distal end portion of the shaft;
a force transmission system coupled to the proximal end portion of the shaft;
a first actuation member extending from the force transmission system along the shaft and coupled to the moveable component, the first actuation member configured to transmit force from the force transmission system to the moveable component; and
a second actuation member extending from the force transmission system along the shaft and coupled to the moveable component, the second actuation member configured to transmit force from the force transmission system to the moveable component,
wherein the force transmission system comprises:
a gimbal rotatable about a first gimbal axis and a second gimbal axis;
a first lever arm comprising a first end portion and a second end portion operably coupled to the gimbal, the first lever arm being configured to rotate about a first pivot axis between the first end portion and the second end portion of the first lever arm; and
a second lever arm comprising a first end portion a second end portion operably coupled to the gimbal, the second lever arm being configured to rotate about a second pivot axis between the first end portion and the second end portion of the second lever arm,
wherein, in response to pivoting of the first lever arm about the first pivot axis, the gimbal rotates about the first gimbal axis,
wherein, in response to pivoting of the second lever arm about the second pivot axis, the gimbal rotates about the second gimbal axis,
wherein rotation of the gimbal about the first gimbal axis results in linear translation of the first actuation member along the shaft, and
wherein rotation of the gimbal about the second gimbal axis results in linear translation of the second actuation member along the shaft.

19. The instrument of claim 18, further comprising a leadscrew, wherein the first end portion of the first lever arm is operably coupled to a leadscrew, the first lever arm pivotable about the first pivot axis in response to rotation of the leadscrew.

20. The instrument of claim 19, wherein the leadscrew is a first leadscrew and the instrument further comprises a second leadscrew, wherein the first end portion of the second lever arm is operably coupled to a second leadscrew and the second lever arm is pivotable about the second pivot axis in response to rotation of the second leadscrew.

21. The instrument of claim 18, wherein the first pivot axis and the second pivot axis are coaxial.

22.-34. (canceled)

35. The instrument of claim 12, wherein:
in response to rotation of the gimbal assembly about the first axis, the first actuation member is linearly translatable relative to shaft and the second actuation member remains substantially stationary; and
in response to rotation of the gimbal assembly about the second axis, the second actuation member is linearly translatable relative to shaft and the first actuation member remains substantially stationary.

36. The instrument of claim 12, wherein the moveable component comprises an articulable structure articulable in pitch and yaw motions.

37. The instrument of claim 12, wherein:
the moveable component is a wrist mechanism coupling an end effector to the distal end portion of the shaft.
